(19) United States
(12) Patent Application Publication
(10) Pub. No.: US 2019/0333616 A1
(43) Pub. Date: Oct. 31, 2019

(54) SYSTEM AND METHOD FOR MEDICAL, HEALTH, WELLNESS AND VETERINARIAN INFOGRAPHIC

(71) Applicant: Landon Pollack, Bell Canyon, CA (US)

(72) Inventor: Landon Pollack, Bell Canyon, CA (US)

(21) Appl. No.: 16/395,196

(22) Filed: Apr. 25, 2019

Related U.S. Application Data
(60) Provisional application No. 62/662,664, filed on Apr. 25, 2018.

(51) Int. Cl. G16H 15/00; G16H 10/60

(52) U.S. Cl. CPC G16H 15/00; G16H 10/20; G16H 10/60

(57) ABSTRACT

In an embodiment, a system and process for a medical infographic is provided. In an embodiment, the method includes receiving raw medical data. The method further includes extracting key details from the medical data. The method also includes associating the key details with data display modules. The method further includes associating references to raw medical data with data display modules. The method also includes displaying data of the data display modules through a user interface. The method further includes receiving a request to modify display. The method also includes modifying display of data of the data display modules in the user interface.

### CLAIM OF PRIORITY

This application claims priority to U.S. Provisional Patent Application No. 62/662,664, entitled “INFOGRAPHIC” and filed on Apr. 25, 2018, which is hereby incorporated herein by reference.

### BACKGROUND

The disclosure relates to acquisition and presentation of information. More particularly the disclosure relates to the acquisition and presentation of medical information.

Medical records are inherently complicated. Presenting them in a manner that is easy to read and can be parsed quickly by both patients and medical professionals is a desirable goal. Doctors, medical providers, patients and caregivers each have different needs and tolerance for display options. Additionally, digging into data or otherwise determining further information can be a challenge with scattered or difficult to parse medical records, for example. Interoperability of data systems and electronic records formats can also make it difficult for many users of different types to deal with legacy systems.

### BRIEF DESCRIPTION OF THE DRAWINGS

The present invention is illustrated by way of example in the accompanying drawings. The drawings should be understood as illustrative rather than limiting.

FIG. 1 is an illustrative example of a medical resume infographic.

FIG. 2 is an illustrative example of an issue summary infographic.

FIG. 3 is an illustrative example of a medical history infographic.

FIG. 4 is an illustrative example of a diagnostic summary infographic.

FIG. 5 is an illustrative example of a labs and providers infographic.

FIG. 6 is an illustrative example of a veterinary infographic.

FIGS. 7, 8 and 9 provide embodiments of systems and devices which may be used with other embodiments presented herein.

FIG. 10 illustrates an embodiment of a method of extraction of medical data.

FIG. 11 illustrates an embodiment of a system for extraction of medical data.

FIG. 12 illustrates an embodiment of a set of data display modules.

FIG. 13 illustrates an embodiment of the set of data display modules of FIG. 12 in a first type of display.

FIG. 14 illustrates an embodiment of the set of data display modules of FIG. 12 in another type of display.

FIG. 15 illustrates an embodiment of the set of data display modules of FIG. 12 in a display undergoing adjustment.

FIG. 16 illustrates an embodiment of a process of displaying a set of data display modules.

FIG. 17A illustrates an embodiment of a system for extraction and display of medical or similar data.

FIG. 17B illustrates an embodiment of a data display module.

FIG. 18 illustrates an embodiment of data displayed from data display modules.

FIG. 19 illustrates an embodiment of a data display module allowing for editing.

FIG. 20 illustrates an embodiment of a data display module in closed and open or expanded form.

FIG. 21 illustrates an embodiment of a data display module in various formats.

FIG. 22 illustrates another embodiment of a data display module in various formats.

FIG. 23 illustrates another embodiment of a set of data display modules for a veterinary application.

FIG. 24 illustrates another embodiment of a data display module.

### DETAILED DESCRIPTION

A system, method and apparatus is provided for a medical infographic. The specific embodiments described in this document represent exemplary instances of the present invention, and are illustrative in nature rather than restrictive.

In the following description, for purposes of explanation, numerous specific details are set forth in order to provide a thorough understanding of the invention. It will be apparent, however, to one skilled in the art that the invention can be practiced without these specific details. In other instances, structures and devices are shown in block diagram form in order to avoid obscuring the invention.

Reference in the specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment, nor are separate or alternative embodiments mutually exclusive of other embodiments.

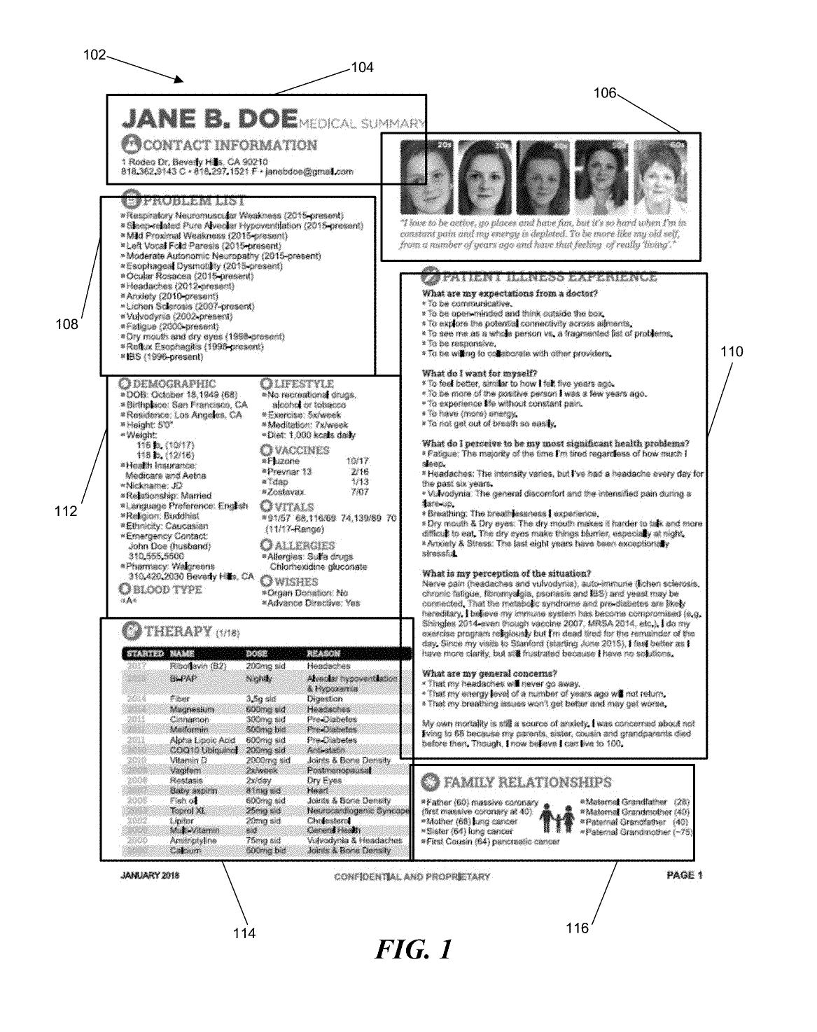

FIG. 1 is an illustrative example of a medical infographic 102. The infographic may be represented as a static report or an editable software file. The infographic appears as a “medical resume” and organizes relevant data into a number of categories. The resume infographic 102 may be used as a coversheet for a patient file and provides an at-a-glance summary of a given patient. This allows doctors new and old to the patient to quickly determine how to approach the patient.

At the top of the resume page is a name and contact section 104. On the upper right corner is a set of progression pictures of the patient 106. The set of pictures may be a combination of historical and generated images, or may be generated using technology associated with age progression processing, for example. The set of pictures further includes a quote that summarizes some aspect of the patient in their own words. The quote or a broader narrative statement may be manually obtained or obtained using natural language processing and artificial intelligence using semantic processing to identify from a disclosure (either written or oral), a passage that describes the way the patient feels. This section may be identified through identification of first person descriptions of what the patient is feeling and/or what they want.

Below the name and contact section 104, is a list of current issues 108. The issues may be obtained via intake or machine analysis of a patient disclosure. Patient disclosure as used herein refers to a set of records about a patient, whether a human or animal subject expected to or actually seeking medical treatment. The set of records, which may include medical, health, wellness and veterinary records in various circumstances and embodiments may have varying levels of completeness and errors or omissions, along with varying formats. References herein to medical records should be understood to refer to the broader categories of medical records, health records, veterinary records, and wellness records, for example, along with other potential repositories of data which may provide for raw data related to care of a patient. Additionally, references to patients should be understood to refer to human patients and animal patients.

The current issues 108 may be augmented or modified by a doctor or other medical provider, for example. Below the set of pictures 106, is an experience summary 110. The experience summary 110 is a concise explanation of how the patient feels and what they hope to achieve through treatment. This is a longer elaboration on the quote positioned by the set of pictures 106. The experience summary 110 includes a description of the expectations of the doctor, the expectations of themselves, what they think is wrong with themselves, and their concerns. This may provide some form of narrative statement about the patient in relation to treatment, for example, and may include material from sources such as a medical provider, patient, or associated third parties such as caregivers, for example. Some of this material may also be hidden, such as in the case of a choice to hide a grim prognosis from a patient prone to giving up hope, for example.

The experience summary 110 is harvested from a written or oral disclosure using text analysis or speech recognition, and semantic natural language processing to identify key words in the disclosure. The keywords are then formatted into a summary “resume” format and plugged into predetermined categories of experience (e.g., those categories listed above).

Below the current issues 108, are demographics and vitals 112. The demographics and vitals are obtained from a patient disclosure, historical medical records, and most recent tests. Such information may include immediately current data obtained as part of treatment in relation to which the medical information is being accessed, or may include historical vitals, for example. Included in the demographics and vitals section 112 is medical statistics pertaining to the patient, lifestyle choices, allergies, vaccinations, and most recent vitals.

Below the demographics and vitals 112 are current treatment regimens 114. The current treatment regimens include medications, supplements and or therapy treatments received by a patient, for example. Next to the current treatments 114 is a family medical history 116, which may provide for something of a medical or health family tree, for example. The family medical history 116 includes causes of death or significant hereditary ailments of the patient's family members, along with information about relationship, age, and other data about such family members. This data is obtained from a patient disclosure and/or previous medical records, for example.

In some embodiments, the infographic is generated in a number of steps including:

Needs HELP; Assessment of needs; Purpose of use; Patient intake; Data collection strategy (sources-providers, hospitals, self, caregivers, family and others, for example); Template selection; Client Homework; Key provider contacts; Data collection outreach; Data aggregation; Development TOC; Data organization (per TOC); Production of Hard and digital file (full PDF medical file by specialty, reverse chronology); Data synthesis; Client 1^{st} review; External review (e.g. PCP, relevant specialist(s), etc.); Internal review; Update; Client 2^{nd} review; Finalize; Dissemination to providers, etc.; Maintenance; and (Bi) Annual update.

In some embodiments, sections of the infographic include:

Contact information; Demographic information; Problem list; Lifestyle; Vaccines; Vitals; Allergies; Blood type; Wishes; Pictures (per decade); Patient goal/hope quote; Patient illness experience; Therapy; Family relationships; Stress summary; Surgery summary; “primary ailment” summary; Medical history summary; Diagnostics summary; Abnormal labs summary; and Providers.

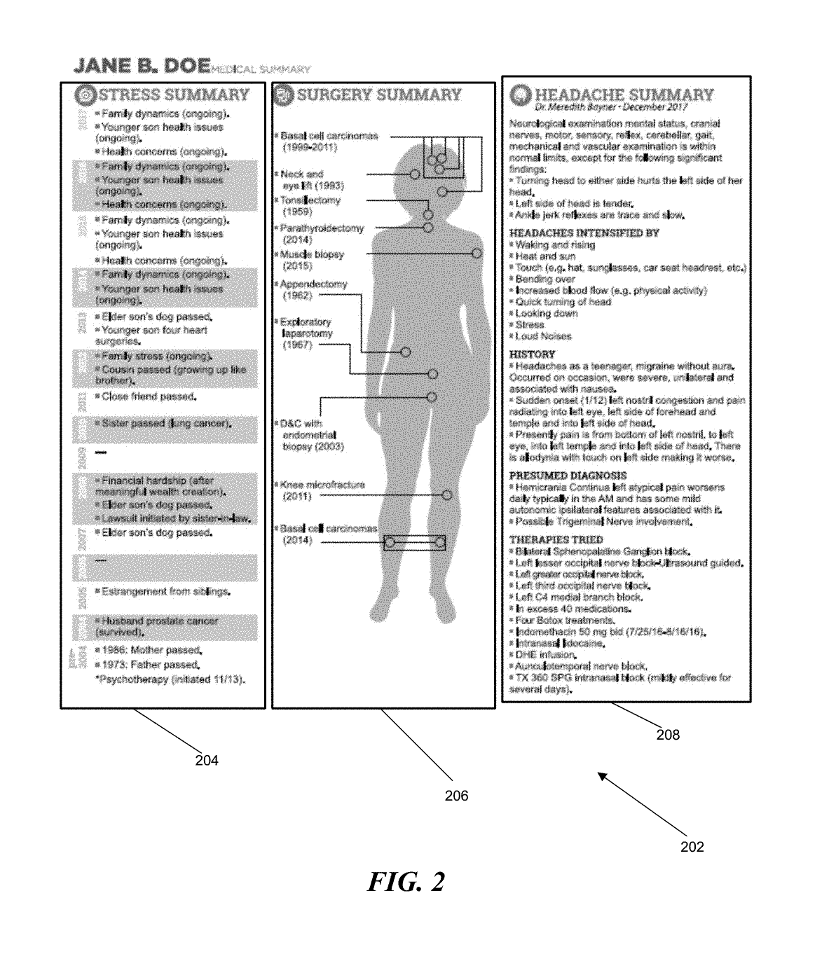

FIG. 2 is an illustrative example of an issue summary infographic 202. The issue summary infographic 202 includes a number of vertical panes 204, 206, and 208. The vertical panes include a history of mental stresses 204. This is obtained from patient disclosure. Data is extracted by identifying causes of mental stress, and tying those to a time period, along with potential analysis of acute or chronic nature of such stress or stress reactions, for example. As with previous infographics, natural language processing is used to identify the mental stresses and associated the timeline. Vertical panes are illustrated as an example, but flexibility of display allows for other formats and expanding or collapsing information as well.

In a second vertical pane is a surgical summary 206. The surgical summary includes a body silhouette identifying which portions of the patient's body have been operated on along with a brief description of the procedure and when the procedure occurred. Other forms of display of such data may also be used, whether in other data display formats or as part of separate data display modules, for example. This information may be extracted from previous medical records and/or patient disclosure. In a third vertical pane, a doctor's report 208 is summarized. The doctor's report may be relevant to the current issues 108 (see FIG. 1). The is obtained from an associated physician or medical care provider involved with treatment and is summarized by natural language processing. In the event of lack of availability of such information, a review of records may be used to attempt to generate such a report, for example.

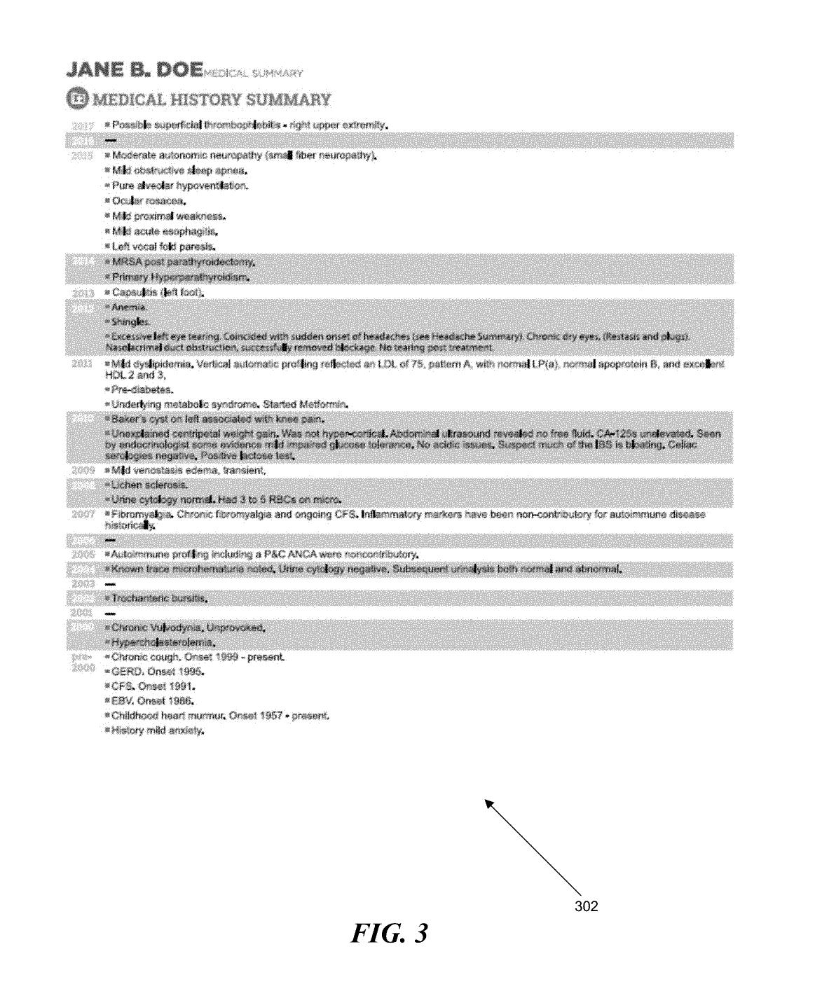

FIG. 3 is an illustrative example of a medical history infographic 302. The medical history infographic 302 is a detailed list of all known symptoms and conditions experienced by the patient listed chronologically, in reverse chronological order in an embodiment. The medical history infographic 302 may span for more than a single page depending on the age and medical experience of the patient. Organization and means for generation of the medical history infographic 302 is similar to the mental stress pane 204 (see FIG. 2) though includes a broader range of medical conditions.

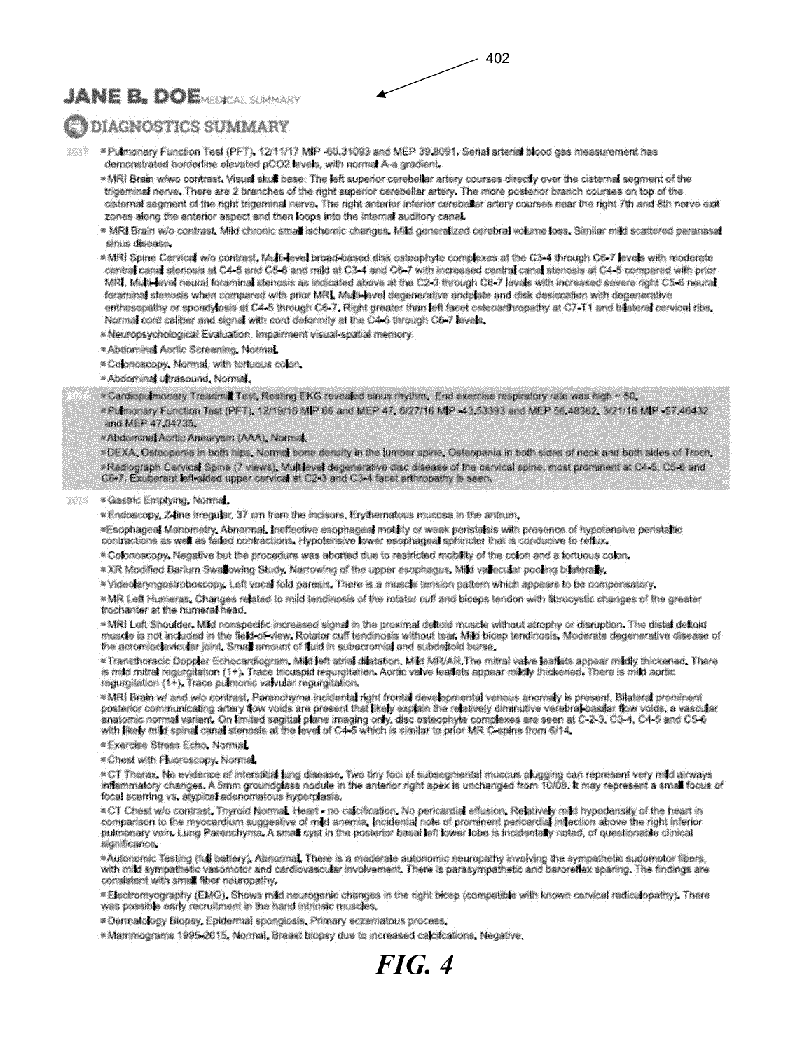

FIG. 4 is an illustrative example of a diagnostic summary infographic 402. The diagnostic summary infographic 402 is a detailed list of all known diagnoses for the patient listed chronologically by time of diagnosis. The diagnostic summary infographic 402 may span for more than a single page or screen, depending on the age and medical experience of the patient. Infographic 402 may also be limited in a hyperfocused application or embodiment, such that size may not be critical to convey important information. Organization and means for generation of the diagnostic summary infographic 402 is similar to the mental stress pane 204 (see FIG. 2) though includes a broader range of medical conditions. The diagnostic summary provided in infographic 402 may be extracted or based on material from other infographics or data display modules, and may allow for correction of data and feedback to other data in a record—correcting an entry may feedback to source data, for example. Moreover, the diagnostic summary of infographic 402 or similar summaries may be expected to link to and allow for traversal of links to information available in other infographics, for example, to allow for deeper dives into data or exploration of underlying data and information, for example.

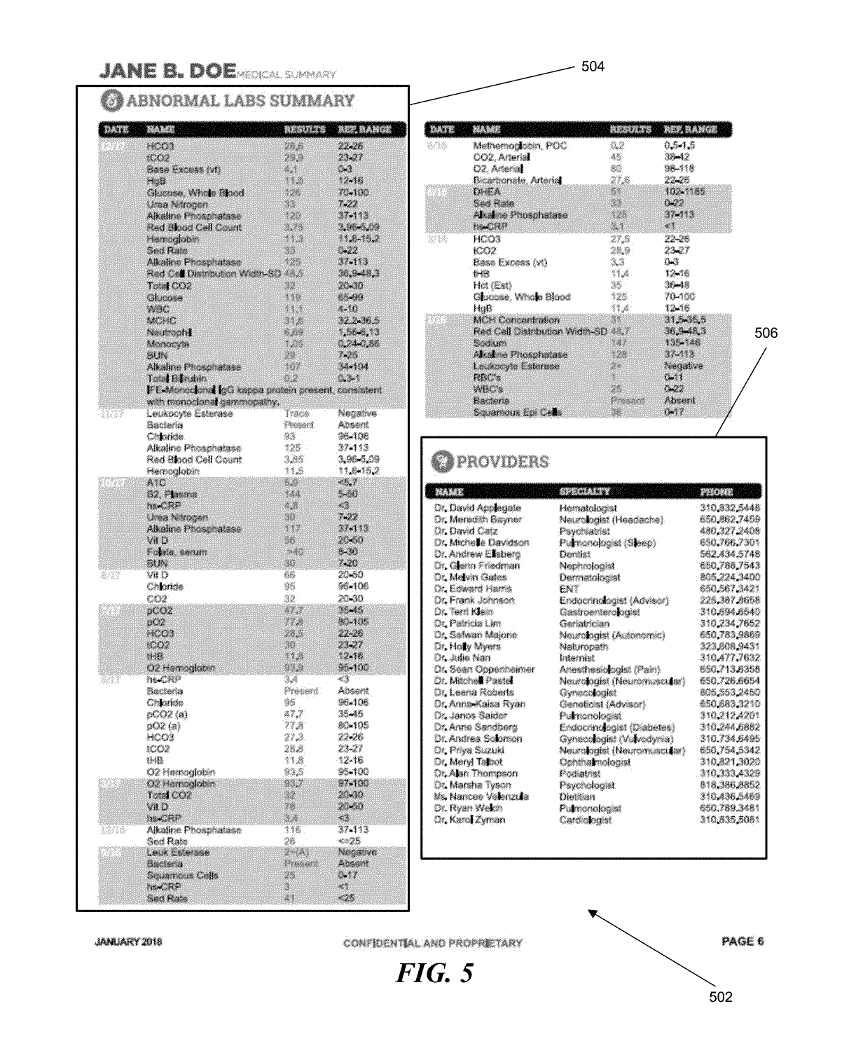

FIG. 5 is an illustrative example of a labs and providers infographic 502. The labs and providers infographic 502 includes a pane that includes a list of tests that have returned with other abnormal results 504 for the patient in conjunction with the date of the tests. Abnormal information can be highlighted in various ways, allowing for viewing or detection of trends or correlations, or absence of such trends or correlations, for example. This information can be extracted from previous medical records. The providers pane 508 is a list of all physicians whom have had contact with the patient, their specialty, and their respective contact information. This too is retrievable from historical medical records. In some embodiments, additional information such as commentary from medical providers or a follow-up or action plan may also be included.

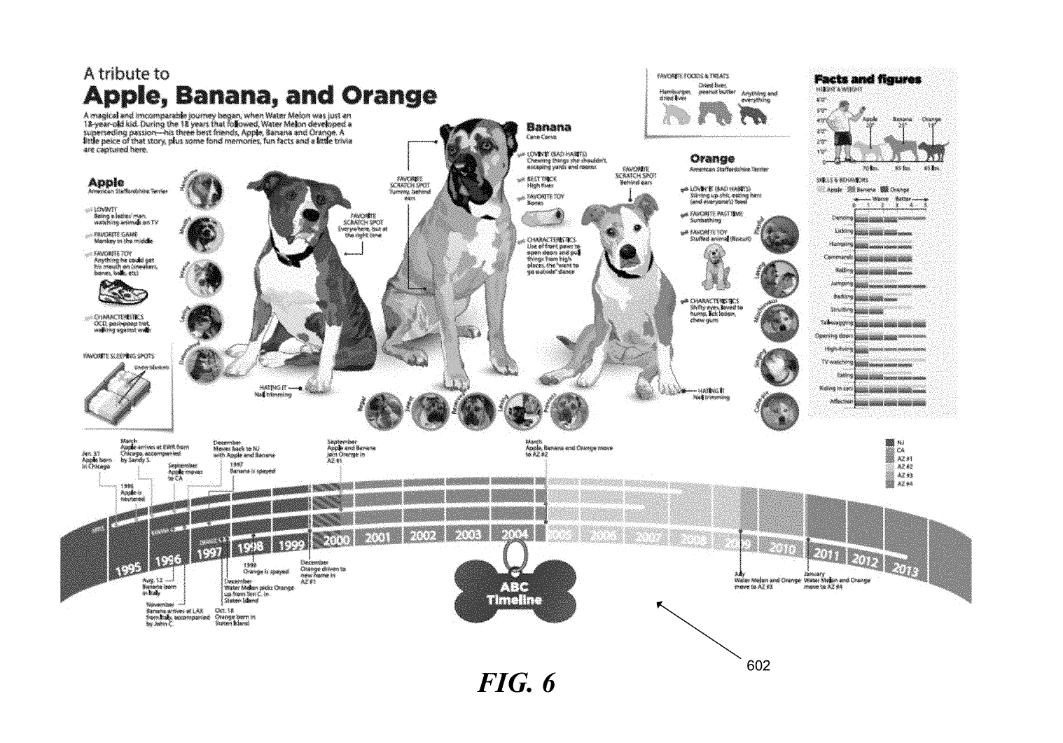

FIG. 6 is an illustrative example of a veterinary infographic 602. The patient need not be human. Further, for pet patients, such as dogs, cats, horses, birds, or other species, other relevant details including calming procedures, and likes or dislikes can be included in order to better serve each pet. Families of pets may be put onto the same infographic, with unique methodology for aggregation. Related to this is showing bread, lineage/family history, adopted vs. purchased, etc. Also items unique to animals include; identification system (tags, license, tattoo, microchip, etc.), types of certifications they animal has received. With animals, social media accounts could be listed as well. Infographics for veterinary applications may be species agnostic, or species specific as well.

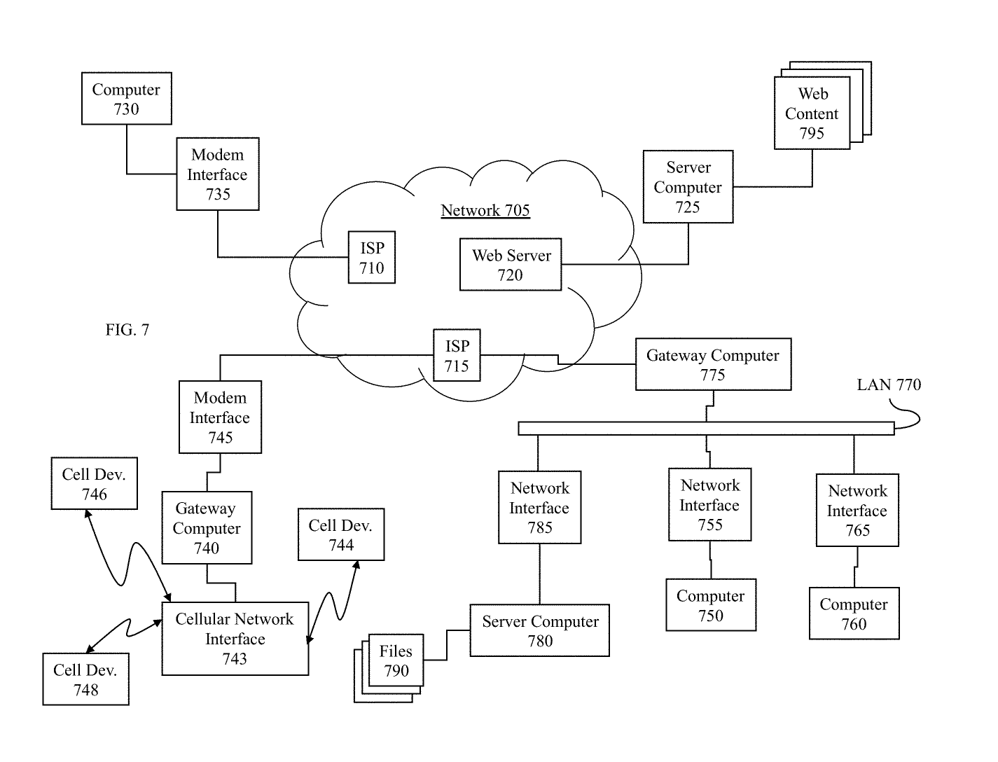

The following description of FIGS. 7-9 is intended to provide an overview of device hardware and other operating components suitable for performing the methods of the invention described above and hereafter, but is not intended to limit the applicable environments. Similarly, the hardware and other operating components may be suitable as part of the apparatuses described above. The invention can be practiced with other system configurations, including personal computers, multiprocessor systems, microprocessor-based or programmable consumer electronics, network PCs, minicomputers, mainframe computers, and the like. The invention can also be practiced in distributed computing environments where tasks are performed by remote processing devices that are linked through a communications network.

FIG. 7 shows several computer systems that are coupled together through a network 705, such as the internet, along with a cellular or other wireless network and related cellular or other wireless devices. The term “internet” as used herein refers to a network of networks which uses certain protocols, such as the TCP/IP protocol, and possibly other protocols such as the hypertext transfer protocol (HTTP) for hypertext markup language (HTML) documents that make up the world wide web (web). The physical connections of the internet and the protocols and communication procedures of the internet are well known to those of skill in the art.

Access to the internet 705 is typically provided by internet service providers (ISP), such as the ISPs 710 and 715. Users on client systems, such as client computer systems 730, 750, and 760 obtain access to the internet through the internet service providers, such as ISPs 710 and 715. Access to the internet allows users of the client computer systems to exchange information, receive and send e-mails, and view documents, such as documents which have been prepared in the HTML format. These documents are often provided by web servers, such as web server 720 which is considered to be “on” the internet. Often these web servers are provided by the ISPs, such as ISP 710, although a computer system can be set up and connected to the internet without that system also being an ISP.

The web server 720 is typically at least one computer system which operates as a server computer system and is configured to operate with the protocols of the world wide web and is coupled to the internet. Optionally, the web server 720 can be part of an ISP which provides access to the internet for client systems. The web server 720 is shown coupled to the server computer system 725 which itself is coupled to web content 795, which can be considered a form of a media database. While two computer systems 720 and 725 are shown in FIG. 7, the web server system 720 and the server computer system 725 can be one computer system having different software components providing the web server functionality and the server functionality provided by the server computer system 725 which will be described further below.

Cellular network interface 743 provides an interface between a cellular network and corresponding cellular devices 744, 746 and 748 on one side, and network 705 on the other side. Thus cellular devices 744, 746 and 748, which may be personal devices including cellular telephones, two-way pagers, personal digital assistants or other similar devices, may connect with network 705 and exchange information such as email, content, or HTTP-formatted data, for example.

Cellular network interface 743 is representative of wireless networking in general. In various embodiments, such an interface may also be implemented as a wireless interface such as a Bluetooth interface, IEEE 802.11 interface, or some other form of wireless network. Similarly, devices such as devices 744, 746 and 748 may be implemented to communicate via the Bluetooth or 802.11 protocols, for example. Other dedicated wireless networks may also be implemented in a similar fashion.

Cellular network interface 743 is coupled to computer 740, which communicates with network 705 through modem interface 745. Computer 740 may be a personal computer, server computer or the like, and serves as a gateway. Thus, computer 740 may be similar to client computers 750 and 760 or to gateway computer 775, for example. Software or content may then be uploaded or downloaded through the connection provided by interface 743, computer 740 and modem 745.

Client computer systems 730, 750, and 760 can each, with the appropriate web browsing software, view HTML pages provided by the web server 720. The ISP 710 provides internet connectivity to the client computer system 730 through the modem interface 735 which can be considered part of the client computer system 730. The client computer system can be a personal computer system, a network computer, a web tv system, or other such computer system.

Similarly, the ISP 715 provides internet connectivity for client systems 750 and 760, although as shown in FIG. 7, the connections are not the same as for more directly connected computer systems. Client computer systems 750 and 760 are part of a LAN coupled through a gateway computer 775. While FIG. 7 shows the interfaces 735 and 745 as generically as a “modem,” each of these interfaces can be an analog modem, isdn modem, cable modem, satellite transmission interface (e.g. “direct PC”), or other interfaces for coupling a computer system to other computer systems.

Client computer systems 750 and 760 are coupled to a LAN 770 through network interfaces 755 and 765, which can be ethernet network or other network interfaces. The LAN 770 is also coupled to a gateway computer system 775 which can provide firewall and other internet related services for the local area network. This gateway computer system 775 is coupled to the ISP 715 to provide internet connectivity to the client computer systems 750 and 760. The gateway computer system 775 can be a conventional server computer system. Also, the web server system 720 can be a conventional server computer system.

Alternatively, a server computer system 780 can be directly coupled to the LAN 770 through a network interface 785 to provide files 790 and other services to the clients 750, 760, without the need to connect to the internet through the gateway system 775.

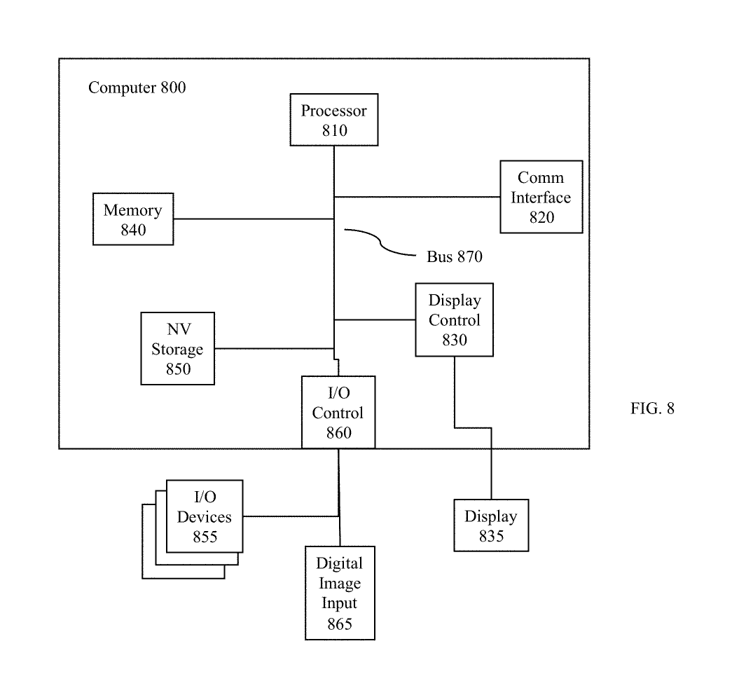

FIG. 8 shows one example of a personal device that can be used as a cellular telephone (744, 746 or 748) or similar personal device, or may be used as a more conventional personal computer, as an embedded processor or local console, or as a PDA, for example. Such a device can be used to perform many functions depending on implementation, such as monitoring functions, user interface functions, telephone communications, two-way pager communications, personal organizing, or similar functions. The system 800 of FIG. 8 may also be used to implement other devices such as a personal computer, network computer, or other similar systems. The computer system 800 interfaces to external systems through the communications interface 820. In a cellular telephone, this interface is typically a radio interface for communication with a cellular network, and may also include some form of cabled interface for use with an immediately available personal computer. In a two-way pager, the communications interface 820 is typically a radio interface for communication with a data transmission network, but may similarly include a cabled or cradled interface as well. In a personal digital assistant, communications interface 820 typically includes a cradled or cabled interface, and may also include some form of radio interface such as a Bluetooth or 802.11 interface, or a cellular radio interface for example.

The computer system 800 includes a processor 810, which can be a conventional microprocessor such as an Intel pentium-based microprocessor or other microprocessor, a Texas Instruments digital signal processor, or some combination of the various types or processors. Memory 840 is coupled to the processor 810 by a bus 870. Memory 840 can be dynamic random access memory (dram) and can also include static ram (sram), or may include FLASH EEPROM, too. The bus 870 couples the processor 810 to the memory 840, also to non-volatile storage 850, to display controller 830, and to the input/output (I/O) controller 860. Note that the display controller 830 and I/O controller 860 may be integrated together, and the display may also provide input.

The display controller 830 controls in the conventional manner a display on a display device 835 which typically is a liquid crystal display (LCD) or similar flat-panel, small form factor display. The input/output devices 855 can include a keyboard, or stylus and touch-screen, and may sometimes be extended to include disk drives, printers, a scanner, and other input and output devices, including a mouse or other pointing device. The display controller 830 and the I/O controller 860 can be implemented with conventional well known technology. A digital image input device 865 can be a digital camera which is coupled to an I/O controller 860 in order to allow images from the digital camera to be input into the device 800.

The non-volatile storage 850 is often a FLASH memory or read-only memory, or some combination of the two. A magnetic hard disk, an optical disk, or another form of storage for large amounts of data may also be used in some embodiments, though the form factors for such devices typically preclude installation as a permanent component of the device 800. Rather, a mass storage device on another computer is typically used in conjunction with the more limited storage of the device 800. Some of this data is often written, by a direct memory access process, into memory 840 during execution of software in the device 800. One of skill in the art will immediately recognize that the terms “machine-readable medium” or “computer-readable medium” includes any type of storage device that is accessible by the processor 810 and also encompasses a carrier wave that encodes a data signal. Alternatively, a physical medium may be used as a machine-readable medium or computer-readable medium.

The device 800 is one example of many possible devices which have different architectures. For example, devices based on an Intel microprocessor often have multiple buses, one of which can be an input/output (I/O) bus for the peripherals and one that directly connects the processor 810 and the memory 840 (often referred to as a memory bus). The buses are connected together through bridge components that perform any necessary translation due to differing bus protocols.

In addition, the device 800 is controlled by operating system software which includes a file management system, such as a disk operating system, which is part of the operating system software. One example of an operating system software with its associated file management system software is the family of operating systems known as Windows® from Microsoft Corporation of Redmond, Wash., and their associated file management systems. Another example of an operating system software with its associated file management system software is the Apple OS® operating system and its associated file management system. The file management system is typically stored in the non-volatile storage 850 and causes the processor 810 to execute the various acts required by the operating system to input and output data and to store data in memory, including storing files on the non-volatile storage 850. Other operating systems may be provided by makers of devices, and those operating systems typically will have device-specific features which are not part of similar operating systems on similar devices. Similarly, WinCÆ or PalmÆ operating systems may be adapted to specific devices for specific device capabilities.

Device 800 may be integrated onto a single chip or set of chips in some embodiments, and typically is fitted into a small form factor for use as a personal device. Thus, it is not uncommon for a processor, bus, onboard memory, and display/I-O controllers to all be integrated onto a single chip. Alternatively, functions may be split into several chips with point-to-point interconnection, causing the bus to be logically apparent but not physically obvious from inspection of either the actual device or related schematics.

Some portions of the detailed description are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The present invention, in some embodiments, also relates to apparatus for performing the operations herein. This apparatus may be specially constructed for the required purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, and each coupled to a computer system bus.

The algorithms and displays presented herein are not inherently related to any particular computer or other apparatus. Various general purpose systems may be used with programs in accordance with the teachings herein, or it may prove convenient to construct more specialized apparatus to perform the required method steps. The required structure for a variety of these systems will appear from the description below. In addition, the present invention is not described with reference to any particular programming language, and various embodiments may thus be implemented using a variety of programming languages.

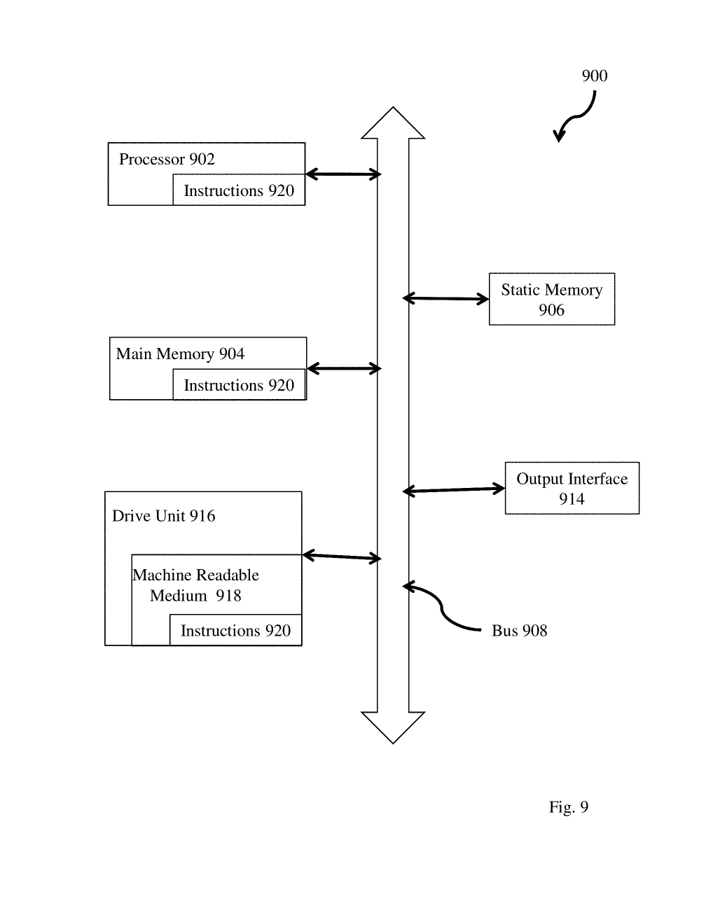

FIG. 9 is a block schematic diagram of a system in the exemplary form of a computer system 900 within which a set of instructions for causing the system to perform any one of the foregoing methodologies and logical flows may be executed.

The computer system 900 includes a processor 902, a main memory 904, and a static memory 906, which communicate with each other via a bus 908. The computer system 900 also includes an output interface 914; for example, a USB interface, a network interface, or electrical signal connections and/or contacts.

The disk drive unit 916 includes a machine-readable medium 918 upon which is stored a set of executable instructions, i.e., software 920, embodying any one, or all, of the methodologies described herein. The software 920 is also shown to reside, completely or at least partially, within the main memory 904 and/or within the processor 902. The software 920 may further be transmitted or received over a network by means of a network interface device 914.

In contrast to the system 900 discussed above, a different embodiment uses logic circuitry instead of computer-executed instructions to implement processing entities. Depending upon the particular requirements of the application in the areas of speed, expense, tooling costs, and the like, this logic may be implemented by constructing an application-specific integrated circuit (ASIC) having thousands of tiny integrated transistors. Such an ASIC may be implemented with CMOS (complementary metal oxide semiconductor), TTL (transistor-transistor logic), VLSI (very large systems integration), or another suitable construction. Other alternatives include a digital signal processing chip (DSP), discrete circuitry (such as resistors, capacitors, diodes, inductors, and transistors), field programmable gate array (FPGA), programmable logic array (PLA), programmable logic device (PLD), and the like.

It is to be understood that embodiments may be used as or to support software programs or software modules executed upon some form of processing core (such as the CPU of a computer) or otherwise implemented or realized upon or within a system or computer readable medium. A machine-readable medium includes any mechanism for storing or transmitting information in a form readable by a machine, e.g., a computer. For example, a machine-readable medium includes read-only memory (ROM); random access memory (RAM); magnetic disk storage media; optical storage media; flash memory devices; electrical, optical, acoustical or other form of propagated signals such as carrier waves, infrared signals, digital signals, etc.; or any other type of media suitable for storing or transmitting information.

Further, it is to be understood that embodiments may include performing operations and using storage with cloud computing. For the purposes of discussion herein, cloud computing may mean executing algorithms on any network that is accessible by internet-enabled or network-enabled devices, servers, or clients and that do not require complex hardware configurations (e.g., requiring cables and complex software configurations, or requiring a consultant to install). For example, embodiments may provide one or more cloud computing solutions that enable users, e.g., users on the go, to access real-time video delivery on such internet-enabled or other network-enabled devices, servers, or clients in accordance with embodiments herein. It further should be appreciated that one or more cloud computing embodiments include real-time video delivery using mobile devices, tablets, and the like, as such devices are becoming standard consumer devices.

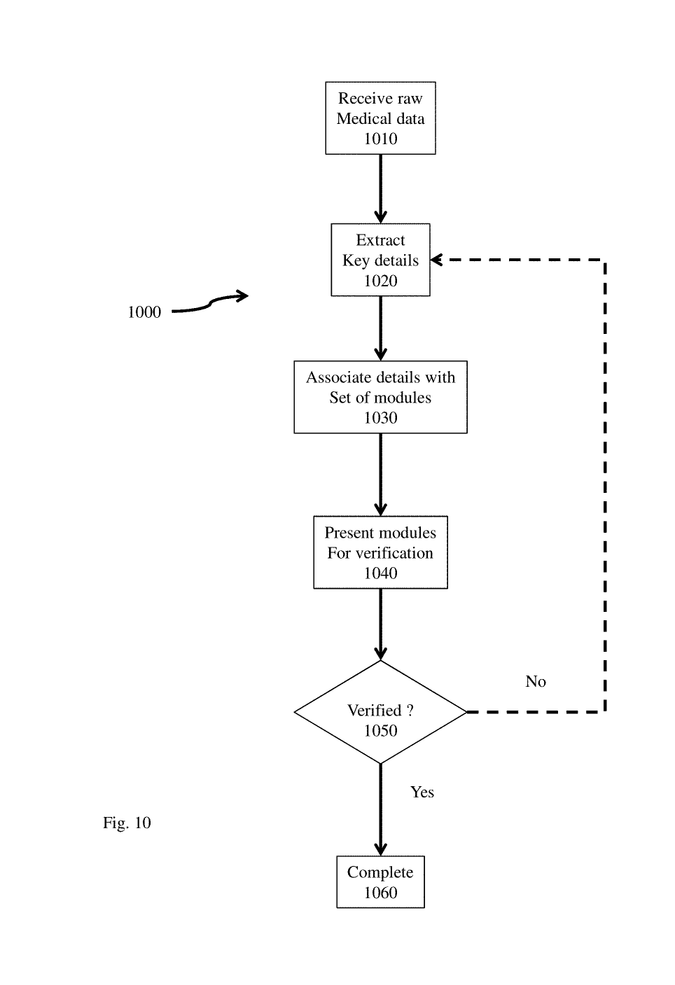

FIG. 10 illustrates an embodiment of a method of extraction of medical data. Process 1000 includes receipt of raw or uncleansed data, extraction of data, association of data with data display modules, preparation for verification, verification, and completion of processing of the data display modules. Process 1000 may be implemented as a series of steps or in a parallel manner, and may be implemented as a set of modules which may be part of a processing system or executed as code in a processing system, for example.

Process 1000 initiates with receipt of raw or uncleansed medical data at 1010. Receipt of such data may be initial receipt of data in forms such as electronic health records, electronic medical records or personal health records, for example. Such data may be provided in a variety of formats and may include errors as a result of previous incorrect entries, omissions, or issues with transfer and saving of data, for example. At 1020, extraction of important or key details of the data received at 1010 occurs. This may involve comparing data with a checklist for a data display module, for example, or review of data by a user of a system to determine such important details for inclusion in one or more data display modules, for example. At 1030, the extracted details are associated with one or more data display modules of a set of data display modules. Such a set of data display modules may be associated with a particular patient, for example. The process may iterate between 1020 and 1030 in order to deal with missed details from the data of 1010, for example, or based on further processing of the data of 1010, for example.

Following preparation of the data display modules, at 1040, the data display modules are presented to a user of a system to allow for verification of data, and potential correction of errors or modification for better display. At 1050, a determination is made as to whether the data of the data display modules has been verified and is potentially ready for display, or needs more processing. The process then returns to 1020 to further extract data if necessary, or completes at 1060. A user of such a system may include a medical provider or staff person, patient, caregiver, holder of power of attorney, or other third party. In some instances and some embodiments, a user may have greater or lesser access rights as a result of the role of the user. Moreover, a user may have an ability or approve or disapprove changes or updates in some instances, to allow for control of information or quality control of changes, for example. Verification at 1050 may be part of such approval or disapproval, for example. Verification at 1050 may also include review to attempt to correct or identify errors, comparison of raw data to extracted data or other verification, for example.

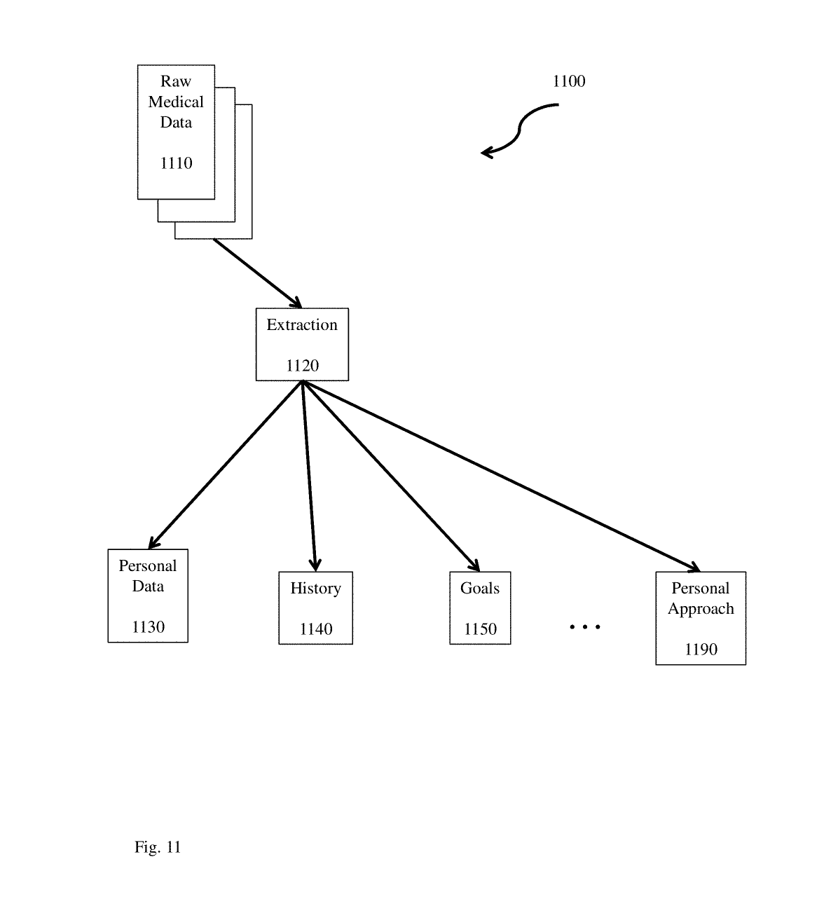

FIG. 11 illustrates an embodiment of a system for extraction of medical, health, wellness and/or veterinarian data. System 1100 may extract data from incoming medical records 1110, such as electronic records for a patient or such as data entered for a patient in system 1100. Extraction engine 1120 may select specific data from records 1110, such as by comparison to checklists or available options for information in a data display module, for example. Extraction engine 1120 may include interactive input from a user of system 1100 in order to further refine such extraction results.

Extraction engine 1120 associates data with data display modules 1130, 1140, 1150 on through 1190, for example. Data display module 1130 may include personal data for a patient, such as name, other identifying information, and health characteristics, for example. Data display module 1140 may include medical history for a patient, for example, such as symptoms, procedures, prescriptions, or other medical history information. Data display module 1150 may include information about goals of a patient, such as weight loss, regaining function (e.g. walking again), specific improved health goals, and other such information, for example. Other data display modules may include a map of symptoms and procedures, for example, or contact information for people associated with a patient, for example. Data display module 1190, for example, may include information useful for personalizing an approach to a patient, such as providing bad news first or telling a joke to set a patient at ease, for example. A potentially infinite set of techniques, examples and other options may be presented for personalizing an approach to a patient.

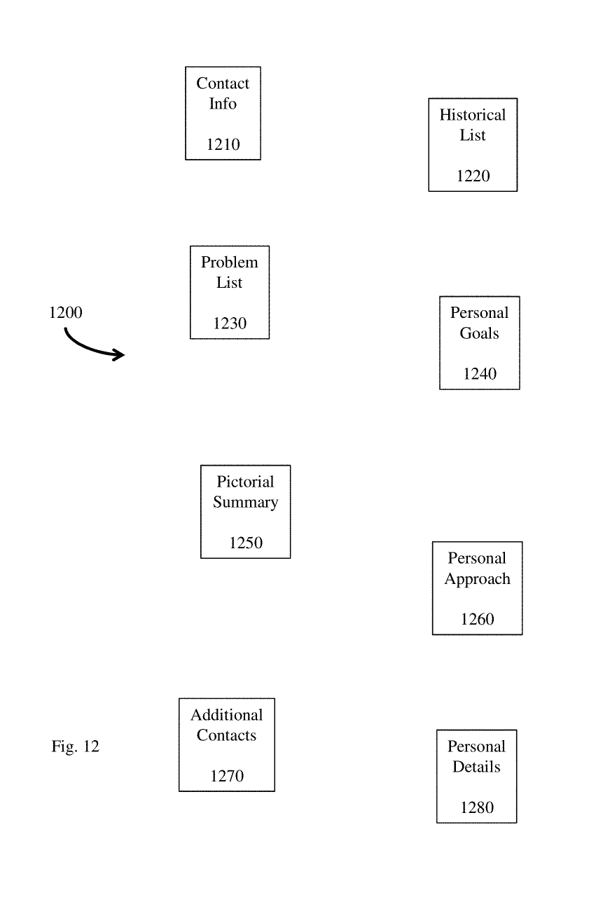

FIG. 12 illustrates an embodiment of a set of data display modules. Data display modules 1200 provide options for display of information extracted from medical records in a variety of formats and with emphasis on different aspects of the data in question. Contact information module 1210 may provide basic information about how to contact a patient and may also include information about medical providers, for example. Historical list module 1220 may provide a list or set of data detailing interactions with medical providers and results therefrom, along with links to further details, for example. Problem list module 1230 may provide types of problems encountered by a patient, for example, including well diagnosed medical problems, potential issues not fully resolved into diagnoses, or symptoms not currently addressed, for example. A problem list may be maintained in multiple ways be multiple modules, with a problem list module 1230 maintaining a specific list, for example. Personal goals module 1240 may provide information about what a patient seeks from care, allowing for a focus on rehabilitation or palliative care, for example, and thereby helping inform medical providers about potential courses of action.

Pictorial summary module 1250 may provide a pictorial summary or map of symptoms, procedures, issues, identifying marks, and other visual representation of medical data, for example. Personal approach module 1260 may provide information about aspects of a patient that can enhance or detract from treatment, such as a fear of medical providers or a very business-like attitude, for example, which may also allow for potential matchmaking, such as choosing or referring to a medical provider based on potential compatibility. Additional contacts module 1270 may provide information about relatives, other providers, holders of a power of attorney, insurance contacts and other similar information, for example. Personal details module 1280 may provide information about a patient such as simple physical characteristics or previously identified details, such as blood type, allergies, aversion to treatment, or other details, for example.

A partial list of potential information to include, wherein information may be included or excluded based on a combination of availability and appropriateness to the patient situation includes:

Demographic information

Problem list

Lifestyle

Vaccines

Vitals

Allergies

Blood type

Wishes

Pictures (per decade or other appropriate interval, either actual or generated, for example)

Patient goal/hope quote

Patient illness experience

Therapy

Family relationships (family medical history or incidents, for example)

Surgery summary

Primary problem summary (a bulleted list, for example)

Medical history summary

Diagnostics summary (often presented at an end traditionally, but may be provided in other areas, such as a headline at the preference of a user, for example)

Emotional/Stress summary or notes, for example

Abnormal laboratory results summary (trends potentially shown in electronic health records, for example)

Providers contacts

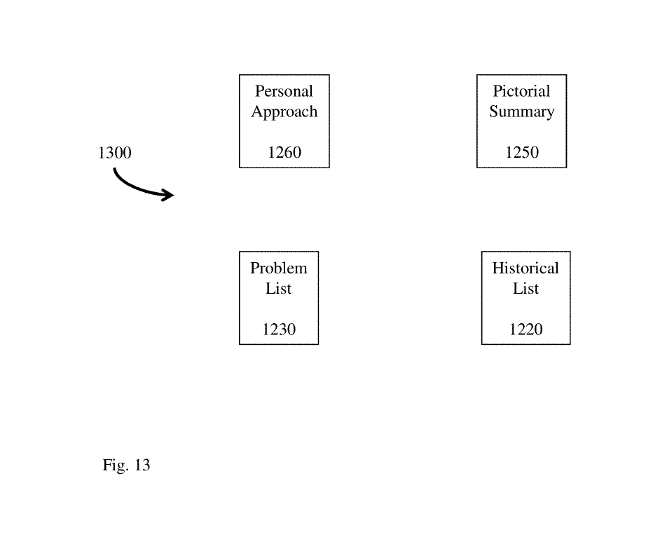

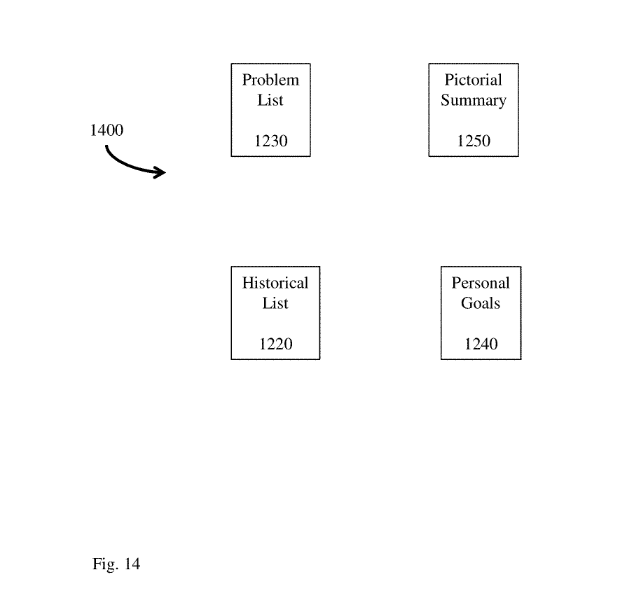

FIG. 13 illustrates an embodiment of the set of data display modules of FIG. 12 in a first type of display. Display 1300 presents information about modules 1260, 1250, 1230 and 1220 in order to provide personal approach data, pictorial summary data, a list of problems and an historical list of medical information, for example. Such a display may be useful to a physician, for example, in reviewing a patient's case and determining how to proceed to a potential diagnosis or treatment, for example. FIG. 14 illustrates an embodiment of the set of data display modules of FIG. 12 in another type of display. Display 1400 includes modules 1230, 1250, 1220 and 1240, to provide information about problems a patient has encountered, a pictorial summary of medical issues, an historical list of medical issues, and personal goals of a patient. Such a display may be useful to a patient to verify that medical information available to a provider appears to be correct and to remind the patient about where a course of treatment may be going, for example.

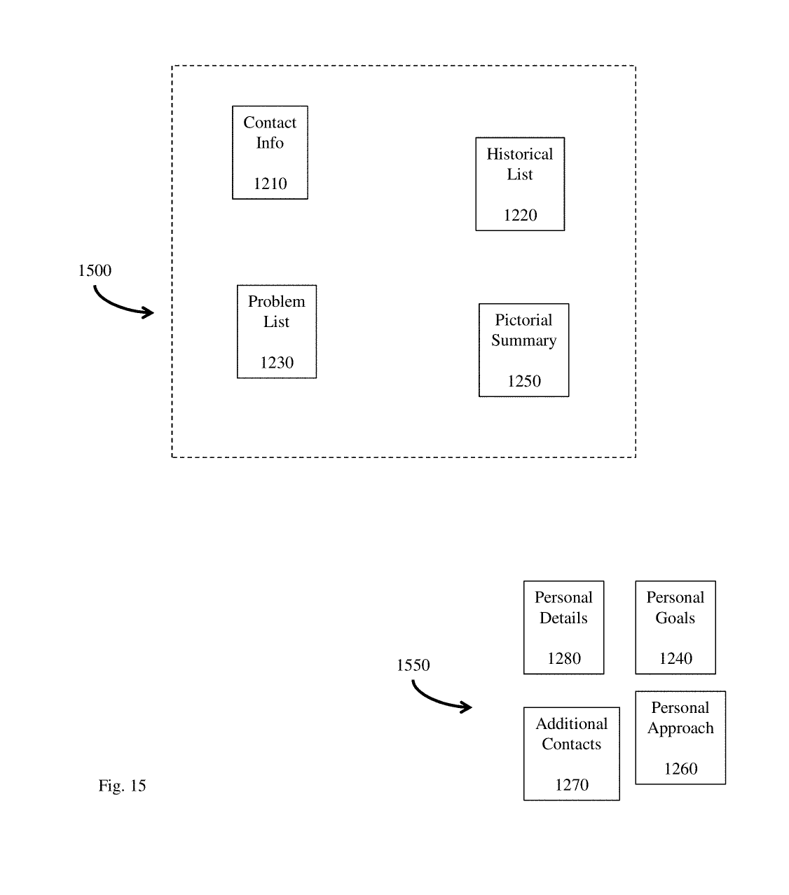

FIG. 15 illustrates an embodiment of the set of data display modules of FIG. 12 in a display undergoing adjustment. Display 1500 includes a set of four data display modules, although more or fewer modules may be included. Other modules 1550 are also available for incorporation into display 1550 at the option of a user of a system interacting with display 1500, for example.

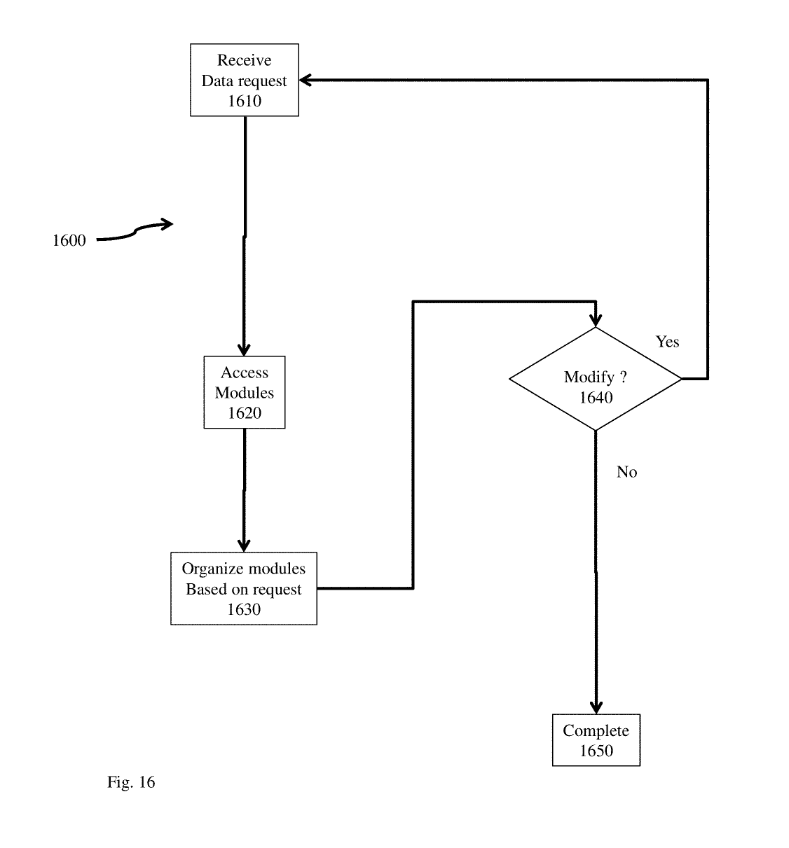

FIG. 16 illustrates an embodiment of a process of displaying a set of data display modules. Process 1600 initiates with a request for data at 1610, such as a request to display data about a patient, for example. At 1620, the process accesses data display modules for information about the patient. At 1630, the process organizes the modules based on predetermined patterns of display of data display modules, such as preferences of a particular user or default displays for types of users, for example, resulting in a display such as that of display 1300 or display 1400, for example. At 1640, the process determines whether modification of the display has been requested. If so, the process returns to 1610 to handle the request for modified display of data, and otherwise, the process may wait for user input or complete at 1650.

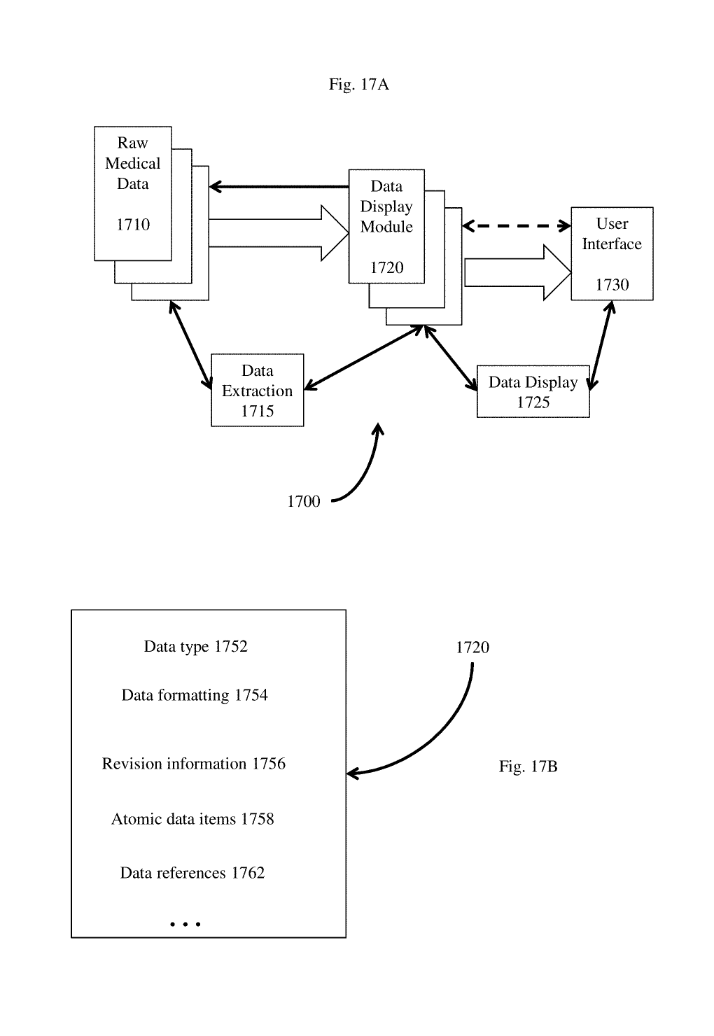

FIG. 17A illustrates an embodiment of a system for extraction and display of medical or similar data. System 1700 includes a data extraction module or engine 1715 and data display module or engine 1725. Raw medical data 1710 is provided in an uncleansed or relatively unprocessed state, and data extraction engine 1715 extracts details and summary information for data display modules 1720. Data display engine 1725 then interacts with data display modules 1720 and user interface 1730 to display data from modules 1720 to a user of user interface 1730.

FIG. 17B illustrates an embodiment of a data display module. Data display module 1720 may include various atomic data information, such as a data type 1752 (e.g. personal data, summary, conveying a type of the data display module 1720), data formatting information 1754 (providing information about the encoding of data or options for displaying data, for example), revision information 1756 (providing indications of when data was updated on an overall or individual basis, for example), atomic data items 1758 (specific data items extracted from raw data and stored for purposes of display), and data references 1762 (indications of where data came from, such as pointers to raw data records, for example).

Raw medical data 1710 may be extracted through an automated process with a data extraction engine 1715 or through an interactive process using engine 1715, for example. Data display modules 1720 may then be populated with atomic data items thus extracted. Data display modules 1720 may further be updated and used in conjunction with user interface 1730 as facilitated by data display engine 1725, which may receive data from data display modules 1720 for display in a variety of formats, such as those shown and described elsewhere in this document.

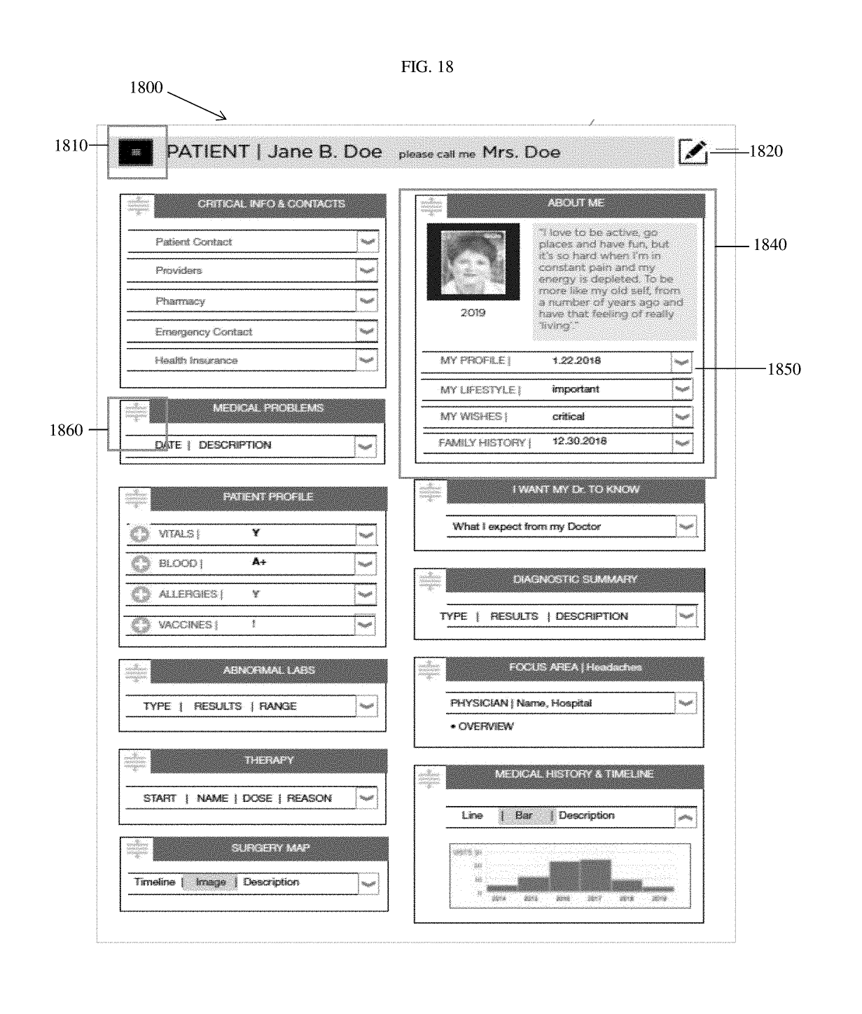

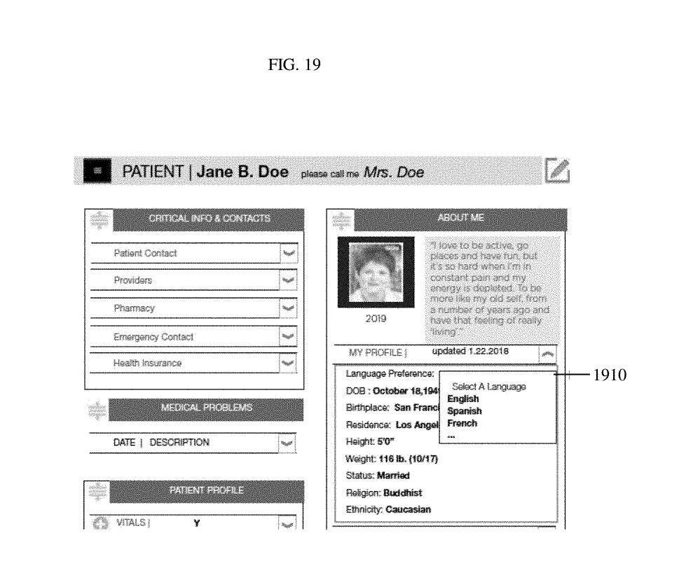

FIG. 18 illustrates an embodiment of data displayed from data display modules. Module 1800 provides a data display module with personal information provided and displayed. Menu 1810 allows for different display options and interaction with an operating system, for example. Edit switch 1820 allows enabling of an editing mode. Personal data display module 1830 provides information about a specific patient as illustrated, and contents 1840 provide the data in readable form. Toggle 1860 allows for opening or closing of a data display module as displayed on a user interface. FIG. 19 illustrates an embodiment of a data display module allowing for editing. Data display module 1910 is opened for editing and a user may enter new or updated information, for example.

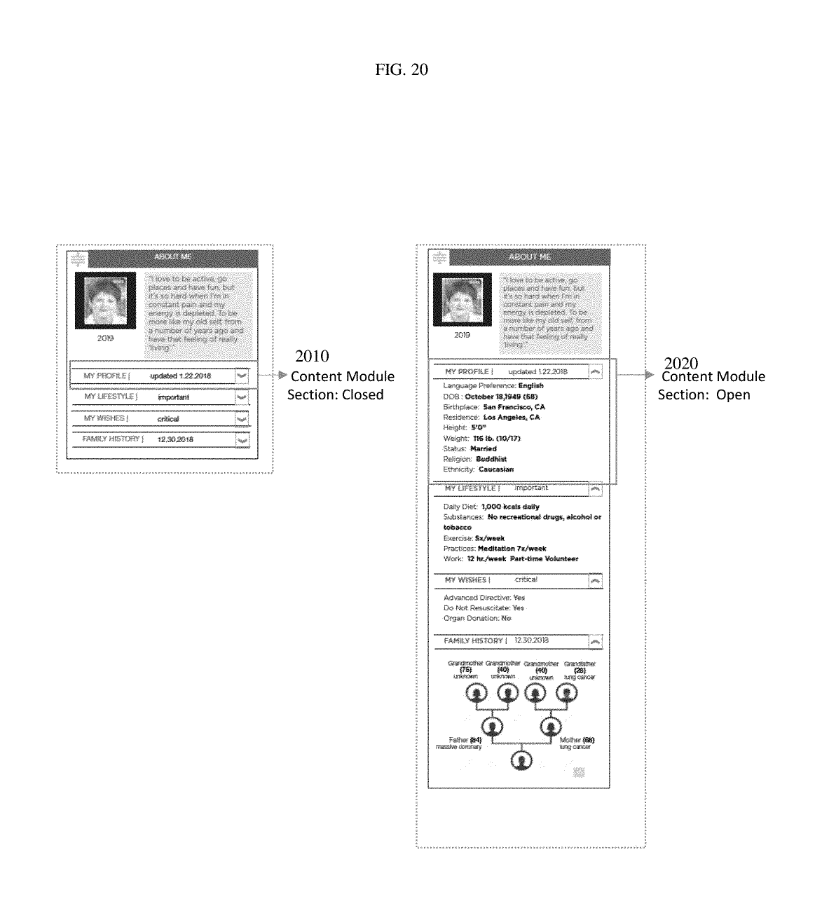

FIG. 20 illustrates an embodiment of a data display module in closed and open or expanded form. Module 2010 is illustrated in a closed form, with limited information displayed. Such a module may be resized, for example, to display more or less information. Module 2020 is the open form of module 2010, where the data display module is displayed in open or expanded form to allow for access to more or all of the data of the data display module, and to allow for resizing and repositioning as with module 2010.

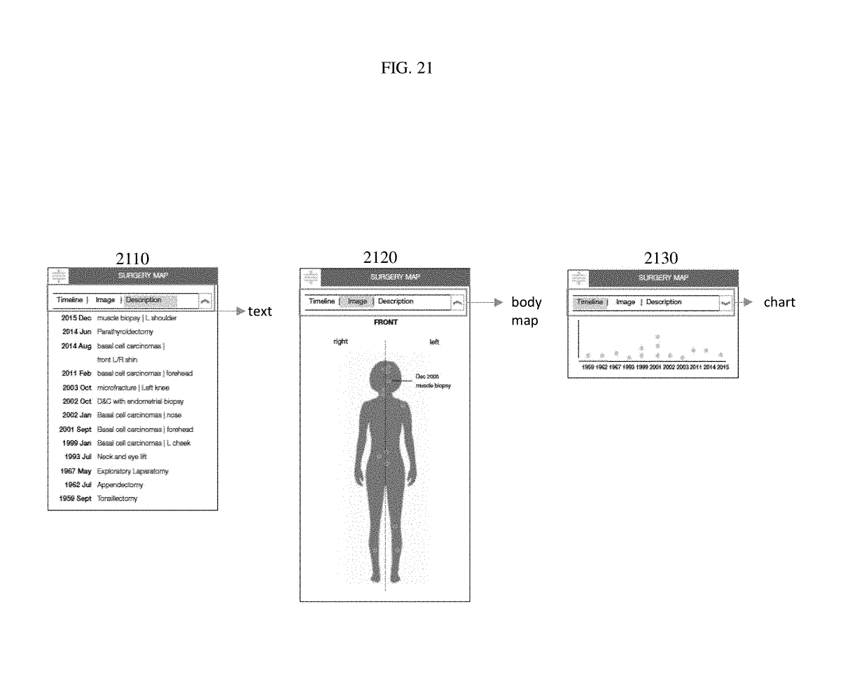

FIG. 21 illustrates an embodiment of a data display module in various formats. Module format 2110 provides a list-based display, such as a reverse chronological list of activity, for example. Module format 2120 provides a map-based display of the data of format 2110. Module format 2130 provides a time-graph or other similar graphic display of incidents over a range of values in chart form. Thus, formats 2110, 2120 and 2130 may all display the same data in different formats.

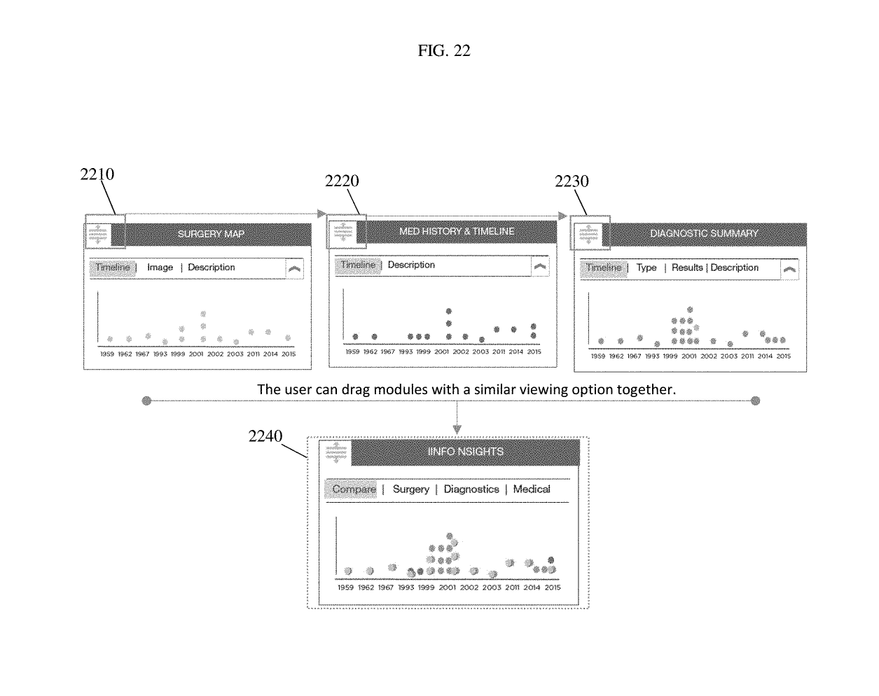

FIG. 22 illustrates another embodiment of a data display module in various formats. Format 2210 provides a display of a first series of data. Format 2220 provides a display of a second series of data. Format 2230 provides a display of a third series of data. Format 2240 provides a display of the first, second and third series of data in a single display, and can be achieved such as through dragging and dropping one or more of formats 2210, 2220 and 2230 together to create a combined display. Such a display as format 2240 may allow for display of multiple time series, for example, and identification of patterns not obvious in each individual series, or other visualization of data.

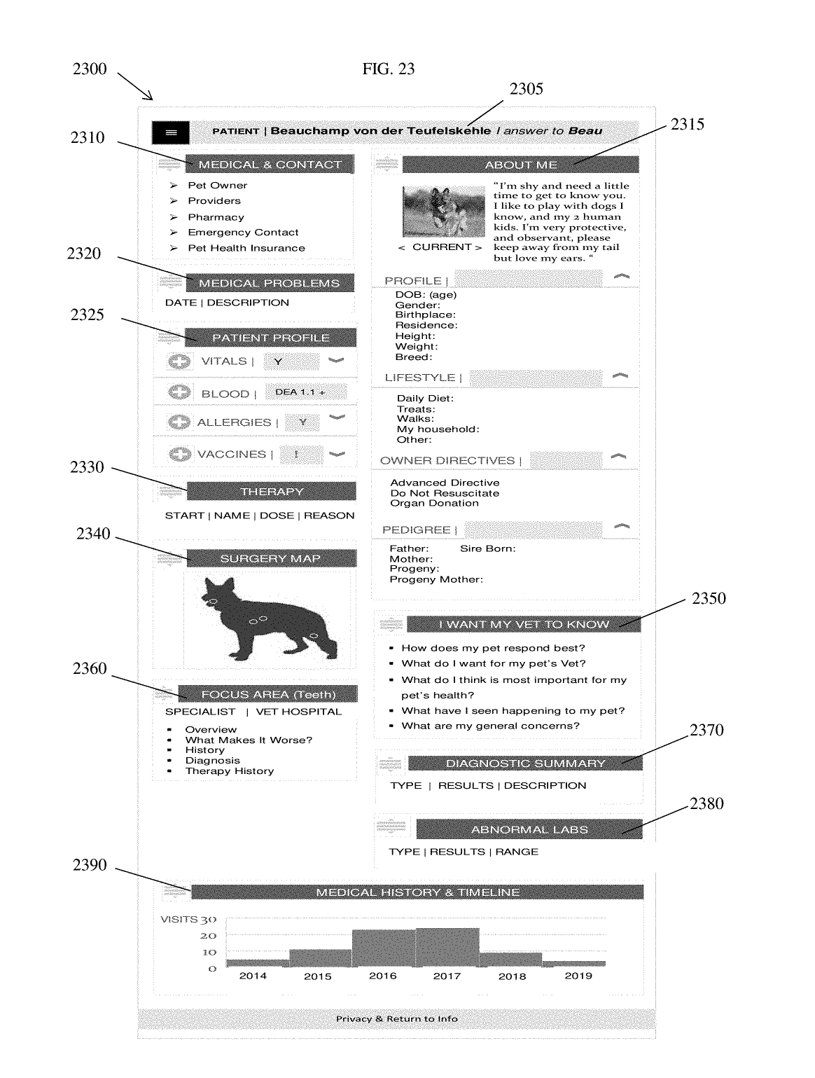

FIG. 23 illustrates another embodiment of a set of data display modules for a veterinary application. Display 2300 combines a variety of data display modules. Module 2310 provides contact information for a pet (e.g. owner and provider information). Module 2320 provides known medical problems. Module 2325 provides a patient profile, such as personal medical information. Module 2330 provides current therapy plans, such as action plans, prescriptions and other similar data. Module 2340 provides a pictorial surgery map which may be implemented or transformed to other display format. Module 2315 provides overall information about a pet, such as personalization information. Module 2350 provides additional specific information for a veterinarian. Module 2360 provides specific information about a focus area of treatment, which may be patient or species specific, or both. Module 2370 provides a summary of diagnostic information. Module 2380 provides highlighted information of abnormal lab results and module 2390 provides a medical history timeline.

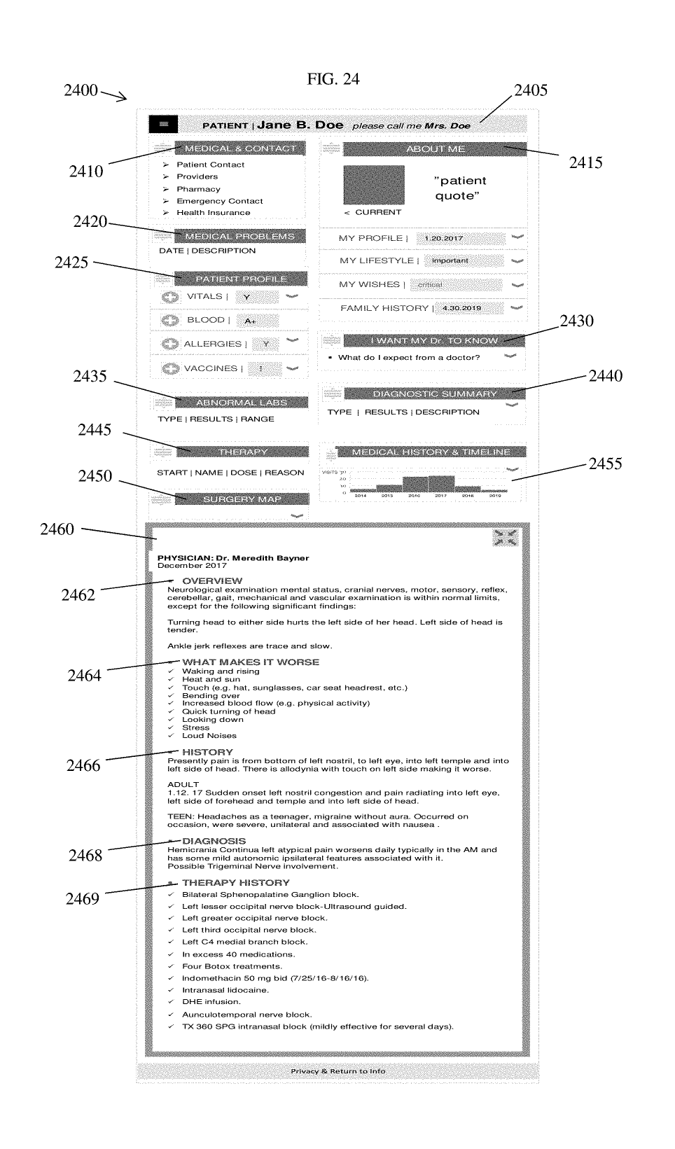

FIG. 24 illustrates another embodiment of a data display module. Display 2400 provides a display of patient information identified for a patient in header 2405. Contact information module 2410 provides contact information. Medical problems module 2420 provides identified problems. Profile module 2425 provides vital health information, for example. Narrative module 2415 provides personal background information. Goals module 2430 provides identified objectives. Abnormal results module 2435 highlights unusual information. Diagnostic and summary module 2440 provides a summary of diagnoses and related information. Therapy module 2445 provides planned and ongoing or prior therapy information. Summary map module 2450 provides a visual representation of information such as surgeries, treatments, or other data. Medical history module 2455 provides historical data about frequency of treatment, for example. Summary map 2460 provides an overview 2462, symptom 2464, history 2466, diagnosis 2466, and therapy history 2468 for a specific problem which a patient or provider may be focusing on.

The systems and methods provided in various embodiments herein provide for a flexible and dynamic system which can display information in a variety of formats which can cater to viewpoints including patients, providers of care, caretakers, staff members, and other third parties. Allowing for flexible display of information potentially helps many users of such data to effectively work with the data, particularly individuals who are not familiar with systems for maintaining medical or other electronic records. This may allow patients and caregivers additional freedom to better determine appropriate courses of treatment or to evaluate options provided by medical professionals, for example. These systems may also allow for visualization of data in a variety of formats which may lead to unexpected insights not generally available from legacy medical records.

The systems and methods may also feature speech-to-text processes allowing for traditional dictation or other verbal or oral entry of information, for example. Moreover, systems may be trained to recognize vernacular phrases associated with conditions which may have substantially different medical terminology, for example. Additionally, patients may be recorded answering questions and allow for audio and/or video playback for other users of such systems and methods, providing for extra information conveyed non-verbally or in a manner not included in text. A patient may record a video answering questions in a questionnaire, for example.

Network effects may also come into play in a variety of ways. Rating systems involving psychometrics may allow for better choices of treatment options based on similar experiences with similarly situated patients, for example. Extracting information from raw data may allow for transition of a human-machine cooperative process to a machine-based process for extraction from raw medical records, for example. Recognition of potential mistakes in data may similarly improve. Patterns from one patient may replicate with other patients on a symptom level as well, allowing for similar efficiencies from data supplied in larger volumes. Additionally, transferability of data and prediction of effects between humans and animals may be improved and utilized in terms of evaluation of experimental treatments or implementation of different strategies, with benefits potentially flowing to both human and animal patients.

The described embodiments are susceptible to various modifications and alternative forms, and specific examples thereof have been shown by way of example in the drawings and are herein described in detail. It should be understood, however, that the described embodiments are not to be limited to the particular forms or methods disclosed, but to the contrary, the present disclosure is to cover all modifications, equivalents, and alternatives.

One skilled in the art will appreciate that although specific examples and embodiments of the system and methods have been described for purposes of illustration, various modifications can be made without deviating from present invention. For example, embodiments of the present invention may be applied to many different types of databases, systems and application programs. Moreover, features of one embodiment may be incorporated into other embodiments, even where those features are not described together in a single embodiment within the present document.

1. A method, comprising:
receiving a set of medical data related to a patient;
extracting summary data of the set of medical data related to a patient;
organizing the summary data into a set of data display modules related to presentation of the summary data;
receiving a request to present the summary data;
and
presenting the summary data using the set of data display modules related to presentation of the summary data in a visual fashion.

2. A method, comprising:
receiving a set of medical data related to a patient;
extracting summary data of the set of medical data related to a patient through:
extracting details of the set of medical data related to a patient;
filtering the details of the set of medical data related to a patient;
organizing the summary data into a set of data display modules related to presentation of the summary data through:
and
linking the details of the set of medical data related to a patient to data display modules of the set of data display modules, wherein the data display modules of the set of data display modules have predetermined characteristics indicating which details of the set of medical data related to a patient are associated with the data display modules of the set of data display modules.

3. A method, comprising:
receiving a request to present summary data of a set of medical data related to a patient;
retrieving data display modules of a set of data display modules related to presentation of the summary data responsive to the request;
presenting the data display modules of the set of data display modules related to presentation of the summary data responsive to the request;
receiving an adjustment request to adjust presentation of the data display modules of the set of data display modules;
changing display characteristics of the data display modules of the set of data display modules responsive to the adjustment request;
and
further presenting the data display modules of the set of data display modules.

4. A system, comprising:
a set of data display modules associated with medical data of a patient, the medical data of the patient organized into categories of data, each data display module of the set of data display modules associated with a category of data;
a user interface coupled to the set of data display modules, the user interface for interacting with data display modules of the set of data display modules, the user interface for displaying data of data display modules of the set of data display modules, the user interface for receiving a request to modify display of data display modules of the set of data display modules, the user interface for displaying data display modules of the set of data display modules in an updated format responsive to a request to modify display of data display modules;
and
a processor for associating data with data display modules of the set of data display modules and accessing data display modules of the set of data display modules and for causing the user interface to display data of data display modules of the set of data display modules.
